(19) United States
(12) Patent Application Publication
(10) Pub. No.: US 2008/0188439 A1
(43) Pub. Date: Aug. 7, 2008

(54) Directed metabolism of compounds by glucuronidation and sulfonation donors to decrease toxicity

(76) Inventors: Steven D. Salhanick, Andover, MA (US); Hilton A. Salhanick, Brookline, MA (US)

Correspondence Address: Mark J. Pandiscio;Pandiscio & Pandiscio, P.C., 470 Totten Pond Road, Waltham, MA, 02451-1914, US

(21) Appl. No.: 11/977,809

(22) Filed: Oct. 26, 2007

Related U.S. Application Data
(60) Provisional application No. 60/854,928, filed on Oct. 26, 2006.

(51) Int. Cl. A61K 31/7076; A61K 31/7072; A61P 1/16

(52) U.S. Cl. 514/47; 514/51

(57) ABSTRACT

Disclosed are compositions and methods of treatment with uridine-based and phosphoadenosine-based cofactors for the prevention and treatment of toxicity associated with compounds such as acetaminophen, which undergo Phase II glucuronidation and sulfonation in the liver. Uridine diphosphoglucose, phosphoadenosine-phosphosulfate, and their derivatives can be administered exogenously either to prevent their depletion in the liver during treatment with acetaminophen, or to replace depleted substrates of UDPGA-transferase or PAPS-transferase in order to treat an individual after a toxic dose of acetaminophen has been ingested.

### REFERENCE TO PENDING PRIOR PATENT APPLICATION

This patent application claims benefit of pending prior U.S. Provisional Patent Application Ser. No. 60/854,928, filed Oct. 26, 2006 by Steven D. Salhanick et al. for DIRECTED METABOLISM OF COMPOUNDS BY GLUCURONIDATION AND SULFONATION COFACTORS TO DECREASE TOXICITY (Attorney's Docket No. 66153P(300476); SAL-1 PROV), which patent application is hereby incorporated herein by reference.

### FIELD OF THE INVENTION

This invention generally relates to preventing or treating liver toxicity associated with the metabolism of chemicals that can undergo glucuronide or sulfonate conjugation. More specifically, this invention relates to compositions and methods to prevent or treat liver toxicity caused by acetaminophen and similar compounds by enhancing metabolism of the compound via glucuronidation or sulfonation.

### BACKGROUND OF THE INVENTION

The normal metabolic pathways in the liver for detoxification and excretion of certain compounds such as acetaminophen can be overwhelmed in cases of acute overdose or prolonged exposure. Certain individuals may have abnormal susceptibility to the toxic effects of normal levels or doses of these agents. In either case, saturation of normal metabolic pathways can result in shunting of metabolism of the compounds through the cytochrome P-450 system in the liver, and lead to the formation of toxic free radical liver inflammation and even liver failure.

Acetaminophen (APAP, N-acetyl-p-aminophenol, paracetamol), solely or in combination with other drugs, is the foremost analgesic drug sold in the United States. By any measure, acetaminophen toxicity is a significant worldwide medical and public health problem (Navarro, V. J. and Senior, J. R., N. Engl. J. Med. 2006; 354:731-739). Acetaminophen is the most common cause of acute hepatic centrilobular necrosis (HCN), a form of liver failure, in the United States, Europe and Australia (Marx, J., Science 2002; 298(5592):341-2; Schmidt, L. E., Hepatology 2002; 35(4):876-882). It causes more hospital admissions for acute liver failure in the United States than any other substance (Gill, R. Q. and Sterling, R. K., J. Clin. Gastroent. 2001; 33:191). Acetaminophen alone or in combination with other analgesics caused at least 171 deaths and 11,692 reports of poisoning of moderate or major toxicity to United States poison centers in 2001 (Litovitz, T. L. et al., Am. J. Emerg. Med. 2002; 20(5):391-452). In England and Wales approximately 500 deaths per year were attributable to acetaminophen overdose between 1993-1997 (Sheen, C. L. et al., QJM 2002; 95(9):609-619). Furthermore, the frequency of acute liver failure induced by excess acetaminophen rose from 28% of all cases in 1998 to 51% in 2003 (Larson, A. M., et al., Hepatology 2005; 42:1364-72). Recently, it was shown that acetaminophen at recommended doses raised alanine aminotransferase (ALT) levels, an indication of hepatic injury if not toxicity (Watkins, P., JAMA 2006; 296:87-93). These findings indicate that the incidence of toxicity at recommended dose ranges may be greater than previously thought. In a study of 54 patients who had ingested an overdose of acetaminophen, 24 patients with histological abnormality had normal liver function tests (Lesna, J. O., et al., Lancet 1975; 1:579-81).

The chemical structure of acetaminophen is shown in FIG. 1. The acetaminophen molecule has two chemical loci of degradation: Carbon 3 of the aromatic ring (Phase I metabolism) and the p-hydroxyl group at Carbon 4 (Phase II metabolism). The different pathways of degradation use different enzymes and involve distinct intermediates. The relative contributions of the major pathways of acetaminophen metabolism in adults are shown in FIG. 2. In one degradation pathway, the hydroxyl group is conjugated to acetaminophen-glucuronide (APAP-G) by glucuronosyl transferase, as shown in FIG. 3. Alternatively, it may be conjugated to acetaminophen-sulfonate (APAP-S) by sulfuryl transferase, as shown in FIG. 4. Furthermore, the ring of the acetaminophen molecule can be oxidized by a cytochrome P-450 enzyme (specifically CYP 2E1) to the toxic free radical N acetyl-p-benzoquinone imine (NAPQI), as shown in FIG. 5. There is no evidence that either APAP-G or APAP-S is toxic; each is rapidly cleared in the urine or bile and reduces the amount of acetaminophen available for CYP 2E1 conversion to toxic products.

Under normal (non-saturated) conditions in an adult, approximately 55% of circulating acetaminophen undergoes Phase II glucuronide conjugation. The glucuronidation pathway for the metabolism of acetaminophen is depicted in FIG. 6, and is discussed in detail in a later section of this document.

In the adult, approximately 30% of circulating acetaminophen undergoes Phase II sulfonate conjugation. The sulfonation pathway in the metabolism of acetaminophen is depicted in FIG. 7. Adenosine phosphosulfate (APS) is transformed to phosphoadenosine phosphosulfate (PAPS) through the action of APS kinase. This compound can then react with acetaminophen via a sulfotransferase enzyme to form acetaminophen-sulfonate (APAP-sulfonate). It is understood that the term sulfonation as used herein is also referred to as sulfation.

Comparing the two major Phase II metabolic pathways, glucuronidation is considered to be a low affinity, high capacity system. In contrast, sulfonation is considered to be a high affinity, low capacity system. Both pathways may be augmented by increased external supply of conjugate donors.

Five percent of circulating acetaminophen normally undergoes Phase I metabolism by cytochrome P-450 enzymes, as shown in FIG. 5. Many drugs are oxygenated or oxidized by the cytochrome P-450 enzyme system. Pharmacokinetic and biochemical data indicate that the cytochrome enzyme subfamily CYP 2E1 is chiefly responsible for the oxidation of acetaminophen in the human (Manyike, P. T., et al. Clin. Pharm. Ther. 2000; 67:275-82). Modeling studies indicate that this enzyme has a rather small substrate binding site; yet it accepts a wide variety of substrates (Lewis, D. F. V., Arch. Biochem Biophys, 2003; 409:32-44; Tan, Y., et al., Xenobiotica, 1997; 27:287-99; Guengerich, F. P., et al., Chem. Res. Toxicol., 1991; 4:168-79). Some of these substrates (e.g., acetaminophen, CCl₄) are converted to free radical products and some (e.g., lauric acid) are converted to conventional P-450 hydroxylated substances. The CYP 2E1 system uses 2 to 3 moles of oxygen to convert one mole of acetaminophen to a toxic intermediate compound, NAPQI, and other free-radical compounds. This compound is claimed to bind to proteins in the hepatic cell membrane and damage the lipid bilayer, leading to hepatic cell injury and death. Necrotic areas in the liver coincide with the distribution of CYP 2E1 and acetaminophen. NAPQI is detoxified by sulfhydryl compounds, such as glutathione (FIG. 5), methionine and N-acetylcysteine (NAC).

Phase I cytochrome P-450 metabolism competes with Phase II metabolic pathways, such as glucuronic acid transferases (FIG. 3) and (to a lesser degree) sulfotransferases (FIG. 4). These Phase II pathways form nontoxic glucuronidated and sulfonated products. The CYP 2E1 oxidation system and the Phase II conjugation systems react with different sites of the acetaminophen molecule. Prior glucuronidation or sulfonation of the hydroxyl group, for example, prevents acetaminophen from binding to the CYP reaction site and, in the unlikely event that it were bound, the stabilization of the hydroxyl by the ester prevents the quinonization and free radical formation. Thus, stabilization glucuronidation or sulfonation of the hydroxyl group on Carbon 4 of acetaminophen may be an important factor in preventing formation of the toxic metabolite NAPQI.

Current treatment of acetaminophen toxicity focuses on detoxification of NAPQI. As shown in FIG. 5, glutathione forms a conjugate with NAPQI, which is then excreted in the urine. Glutathione can become depleted in the setting of large concentrations of acetaminophen. Acetaminophen toxicity is currently treated by administering compounds such as N-acetylcysteine (NAC) that are thought to contribute sulfhydryl groups to make up for the depletion of glutathione. It has been shown that the incidence of liver toxicity can be reduced if NAC is administered when levels of acetaminophen exceed 150 μg/mL at 4 hours after acetaminophen ingestion (Smilkstein, et al., New Engl. J. Med., 1988; 319:1557-1562). A more recent study recommends initiation of treatment at lower levels of plasma acetaminophen (Kozer, E., Koren, G., Drug Saf., 2001, 24:503-612).

However, a recent study raises questions about the mechanism by which glutathione substitutes exert their beneficial effects. Mice deficient in glutathione S-transferase Pi, an enzyme that catalyzes the conjugation of glutathione with NAPQI, were found to be resistant to the hepatotoxic effects of acetaminophen (Henderson, C. J., et al., Proc. Nat. Acad. S., 2000; 97:12741-45). No differences were found between the wild-type mice and the enzyme-deficient mice with respect to cytochrome enzyme concentrations, acetaminophen metabolism, glutathione and glucuronide conjugates in urine and bile, and covalent binding of NAPQI. Resting levels of glutathione were similar and were rapidly depleted, although less so in the enzyme-deficient animals. Thus, glutathione conjugation with NAPQI may not be the primary mechanism by which an individual is protected from liver damage. Postulated mechanisms for NAC's effectiveness, for example, include improvement of liver blood flow, glutathione replenishment, decreased cytokine production and scavenging of free radicals.

Other putative NAPQI scavengers or CYP inhibitors have included propylene glycol (U.S. Pat. No. 4,307,073), methionine sulfoxide (U.S. Pat. No. 4,314,989), L-cysteine (U.S. Pat. No. 5,716,991), diallyl sulfide, diallyl sulfone, and other organosulfur compounds (U.S. Pat. No. 5,474,757). None of these substances achieved commercial success, either because of clinical ineffectiveness or because of unacceptable side effects.

Other than improved supportive care and liver transplantation, therapy for acetaminophen poisoning has not changed significantly in the last 20 years (Bizovi, K. E., Acetaminophen, in Goldfrank's Toxicologic Emergencies, NY, McGraw-Hill, 2002; Navarro, V. J., Senior, J. R., New Engl. J. Med. 2006; 354:731-39). Survival rates of patients with severe toxicity have shown little improvement over the years (Lee, W. M., New Engl. J. Med. 2003; 349:474-85). Current opinion holds that acetaminophen and other potential hepatotoxins are metabolized by cytochrome P-450 systems to NAPQI and possibly other toxic free radicals. These radicals bind covalently with glutathione and sulfhydryl groups of vital protein(s) or interfere with a vital system. Acetaminophen toxicity is associated with biochemical changes that lead to hepatic centrilobular necrosis (HCN). Pathologic and physiologic studies have shown that early mitochondrial dysfunction is a prominent finding in acetaminophen toxicity, consistent with a lack of available oxygen at the cellular level following poisoning. It is thought that NAPQI results in oxidation of enzymes and the impairment of cellular defense mechanisms, calcium accumulation, lipid peroxidation, DNA fragmentation, mitochondrial injury, cellular apoptosis, inflammatory responses to cellular necrosis, and impaired microcirculation and oxygenation. Mitochondrial swelling, an indication of mitochondrial dysfunction, is present 15-60 minutes after exposure to toxic doses of acetaminophen (600 mg/kg in mice) (Walker, R. M., et al., Laboratory Investigation 1980; 42:181-9; Placke, M. E., et al. Toxicologic Pathology 1987; 15:431-8; Ruepp, S. U., et al., Toxicological Sciences 2002; 65:135-50). Decreased mitochondrial respiration in response to acetaminophen poisoning and NAPQI exposure has been demonstrated repeatedly under a variety of experimental conditions (Meyers, L. L., et al., Toxicol. & Appl. Pharmacol. 1988; 93(3):378-87; Esterline, R. L., Ji, S., Biochem. Pharmacol. 1989; 38(14):2390-92; Esterline, R. L., et al., Biochem. Pharmacol. 1989; 38(14):2387-90; Katyare, S. S., Satav, J. G., Brit. J. Pharmacol. 1989; 96(1):51-58; Ramsay, R. R., et al., Arch. Biochem. & Biophysics 1989; 273(2):449-57; Andersson, B. S., et al., Chemico-Biological Interactions 1990; 75(2):201-11; Burcham, P. C., Harman, A. W., J. Biol. Chem. 1991; 266(8):5049-54; Burcham, P. C., Harman, A. W., Toxicology Letters 1990; 50(1):37-48; Strubelt, O., Younes, M., Biochem. Pharmacol. 1992; 44(1):163-70; Donnelly, P. J., et al., Arch. Toxicol. 1994; 68(2):110-18; Parmar, D. V., et al., European J. Pharmacol. 1995; 293(3):225-9). Both acetaminophen and NAPQI, applied directly to isolated mitochondria in high concentrations, decrease respiration rapidly (Ramsay, R. R., et al., Arch. Biochem. & Biophysics 1989, 273(2):449-57).

In vivo, a toxic dose of acetaminophen can affect mitochondrial function within 15 minutes to one hour. Glutathione is depleted approximately 20 minutes after acetaminophen administration (Jaeschke, H., J. Pharmacol. & Exp. Ther. 1990; 255(3):935-41). Intracellular calcium concentration starts to rise approximately 1 hour after acetaminophen poisoning (Moore, M., et al., J. Biol. Chem. 1985; 260(24)13035-40).

The pharmacokinetics of APAP have been well-studied in the human. The gastrointestinal tract absorbs acetaminophen rapidly, and maximal blood concentrations are found at 20-80 minutes after ingestion, depending on tablet formulation and other factors that influence absorption (Forrest, J. A., et al., Clin. Pharmacokinetics 1982; 7(2):93-107). The experimental half-life (t_{0.5} β) of a single oral dose of 1-3 gms is about 105-160 minutes (Id.). However, after an overdose of acetaminophen, the median half-life in 112 patients was noted to be 5.4 hours (Schiodt, F. V., et al., Clin. Pharm. & Ther. 2002; 71(4):221-5). The volume of distribution for acetaminophen is approximately 0.95 L/Kg, i.e., tissue and plasma drug concentrations are almost equal (Forrest et al. 1982). Given that 95% of a therapeutic dose of acetaminophen is rapidly biotransformed in the liver, it is evident that initial hepatic concentrations are relatively high. Studies suggest that a blood concentration of acetaminophen of 300 μg/ml (2 mM) places an individual at high risk of hepatotoxicity (Rumack B. H., Matthew, H., Pediatrics 1975; 55(6):871-6; Lee, W. M., New Engl. J. Med. 2003; 349(5):474-85). Circumstances leading to a longer acetaminophen elimination half-life, presumably due to saturation of detoxification systems, result in greater toxicity in both NAC treated and untreated patients (Prescott, L. F., et al., Lancet 1971; 1(7698):519-22; Schiodt, F. V., et al., Clin. Pharm. & Ther. 2002; 71(4):221-5).

Plasma levels of conjugated metabolites exceed those of acetaminophen within 2 hours after therapeutic dosing and follow similar elimination kinetics, attesting to the rapid rates of conversion of acetaminophen in the liver (Forrest, J. A., et al., Clin. Pharmacokinetics 1982; 7(2):93-107). It is generally accepted that over 90% of the toxic dose is excreted in the first 24 hours (Id.; Slattery, J. T., et al., Clin. Pharm. & Ther. 1987; 41(4):413-18). Studies from controlled animal experiments suggest that most of the toxic effects are initiated within the first half-life (about 3 hours) depending on dose (See Zimmerman, H. J., Hepatotoxicity. The Adverse Effects Of Drugs And Other Chemicals On The Liver, Philadelphia, Lipincott Williams and Wilkins, 1999). The pattern of changes of metabolites may extend over several days (Id.).

Thus, it is clear that improved methods of preventing and treating acetaminophen toxicity are needed. A desirable approach is to provide enough Phase II cofactors to limit the formation of the cytochrome-mediated toxic products in the first instance, either preventatively by administering the cofactors or precursors thereof with each dose of acetaminophen or therapeutically by administering the cofactors or precursors thereof as soon as possible after a toxic dose of acetaminophen has been taken.

### SUMMARY OF THE INVENTION

This invention generally relates to the prevention and treatment of toxicity caused by acetaminophen and other drugs that are metabolized in similar ways. Unexpectedly, it has been found that treatment with compositions of Phase II hepatic detoxification cofactors, uridine diphosphoglucose, its precursors, salts, acids or mixtures thereof, and/or phosphoadenosine phosphosulfate (PAPS), its precursors, salts, acids or mixtures thereof, used alone or in combination with other pharmaceutical compositions, can reduce the incidence and degree of liver toxicity caused by chemical compounds susceptible to glucuronidation in the liver.

Among other things, unexpectedly, it has been found that treatment with a glucuronidation donor, and/or a sulfonation donor, alone or in combination with other pharmaceutical compositions, can reduce the incidence and degree of liver toxicity caused by chemical compounds susceptible to conjugational inactivation in the liver.

In a preferred form of the invention, prevention or treatment of acetaminophen toxicity is effected by a glucuronidation donor or a precursor thereof, or the sulfonation donor or a precursor thereof, or a combination thereof.

More particularly, in a preferred form of the invention, there is provided a pharmaceutical composition comprising uridine diphosphoglucose (UDPG), uridine diphosphoglucuronic acid (UDPGA), uridine diphosphate (UDP) or uridine triphosphate (UTP), and other salts or mixtures thereof, and/or PAPS, phosphoadenosine phosphate (PAP) or phosphoadenosine (PA), in combination with acetaminophen. These compositions may be in oral form, as a rectal or vaginal suppository, a transdermal patch, or a parenteral formulation. Oral forms of the composition can include a tablet, a capsule, a gel, a wafer, a chewable tablet, a caplet, melt-away, buffered effervescent granules, a solution, a lyophilized solution, a suspension, a lyophilized suspension, a syrup and an elixir. The dose of any of these compositions can be in the range of about 0.1 to 10, or 0.5 to 2 moles per mole of acetaminophen ingested. The dose may even be about 1 mole per mole of acetaminophen ingested.

The composition can be administered as part of a combination therapy with other detoxifying compounds, including, for example, N-acetylcysteine, methionine, inhibitors of cytochrome P-450 metabolism or cofactors of sulfonation.

A preferred form of the invention also encompasses a method of treating acetaminophen-associated toxicity in a person. A composition containing a therapeutically effective amount of UDPG, UDPGA, UDP, UTP or other salts or mixtures thereof, or PAPS, PAP, PA or other salts or mixtures thereof, is administered to the person or patient in need thereof. The acetaminophen-associated toxicity can be due to a variety of factors such as an acute overdose of acetaminophen, the ingestion of a plurality of doses of acetaminophen, or abnormal susceptibility to acetaminophen toxicity. Examples of abnormal susceptibility to acetaminophen toxicity can include, but are not limited to, genetic predisposition to liver disease, chronic ethanol ingestion, chronic acetaminophen ingestion, concurrent use of other drugs, exposure to toxic chemicals, fasting, hyperthyroidism, cirrhosis, hepatitis, cholangitis, tumors of the liver, liver transplantation, ischemia of the liver, and heart failure.

The method of treatment can include a combination therapy of UDPG, UDPGA, UDP, UTP or other salts or mixtures thereof and/or PAPS, PAP, PA or other salts or mixtures thereof with NAC, methionine, other glutathione donors, inhibitors of cytochrome P-450 metabolism. Treatment can be administered in a parenteral, oral or rectal form. Treatment is normally administered within 24 hours, or preferably within about 4 hours following acetaminophen ingestion, and can be administered for a period of time ranging from about 24 hours to about 72 hours. The treatment can be administered continuously, for example, by continuous intravenous or enteral infusion, or, alternatively, the treatment can be administered in a plurality of discrete doses.

This invention also encompasses a method of preventing acetaminophen-associated toxicity in a person taking acetaminophen by administering a composition containing a therapeutically effective amount of UDPG, UDPGA, UDP, UTP or other salts or mixtures thereof, or PAPS, PAP, PA or other salts or mixtures thereof. The composition can be administered in the form of a tablet, capsule, gel wafer, chewable tablet, caplet, melt-away, buffered effervescent granules, solution, lyophilized solution, suspension, lyophilized suspension, syrup or elixir.

In one preferred form of the present invention, there is provided a pharmaceutical composition comprising (i) a xenobiotic substance that is hydroxylated or can be hydroxylated and (ii) a glucuronidation donor for yielding a xenobiotic glucuronide, wherein the glucuronidation donor comprises at least one from the group consisting of: uridine diphosphoglucuronic acid (UDPGA); precursors thereof; salts thereof; and mixtures thereof. And in one particularly preferred form of the invention, the xenobiotic substance comprises acetaminophen and the xenobiotic glucuronide comprises acetaminophen glucuronide (APAP-G).

In another preferred form of the present invention, there is provided a pharmaceutical composition comprising (i) a xenobiotic substance that is hydroxylated or can be hydroxylated and (ii) a sulfonation donor for yielding a xenobiotic sulfonate, wherein the sulfonation donor comprises at least one from the group consisting of: phosphoadenosine-phosphosulfate (PAPS); precursors thereof; salts thereof; and mixtures thereof. And in one particularly preferred form of the invention, the xenobiotic substance comprises acetaminophen and the xenobiotic sulfonate comprises acetaminophen sulfonate (APAP-S).

In still another preferred form of the present invention, there is provided a method for preventing liver damage in a patient caused by the ingestion of a xenobiotic substance that is hydroxylated or can be hydroxylated, comprising administering a therapeutically effective dose of a glycuronidation donor to the patient, wherein the glucuronidaton donor comprises one from the group consisting of: uridine diphosphoglucuonic acid (UDPGA); precursors thereof; salts thereof; and mixtures thereof. And in one particularly preferred form of the invention, the xenobiotic substance comprises acetaminophen.

In yet another preferred form of the present invention, there is provided a method for preventing liver damage in a patient caused by the ingestion of a xenobiotic substance that is hydroxylated or can be hydroxylated, comprising administering a therapeutically effective dose of a sulfonation donor to the patient, wherein the sulfonation donor comprises one from the group consisting of: phosphoadenosine-phosphosulfate (PAPS); precursors thereof; salts thereof; and mixtures thereof. And in one particularly preferred form of the invention, the xenobiotic substance comprises acetaminophen.

For the purposes of the present invention, it should be appreciated that the term “xenobiotic substance that is hydroxylated or can be hydroxylated” is intended to include xenobiotic substances which may be hydroxylated either chemically prior to use or biologically by body enzymes (in vivo).

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 shows the chemical structure of acetaminophen;

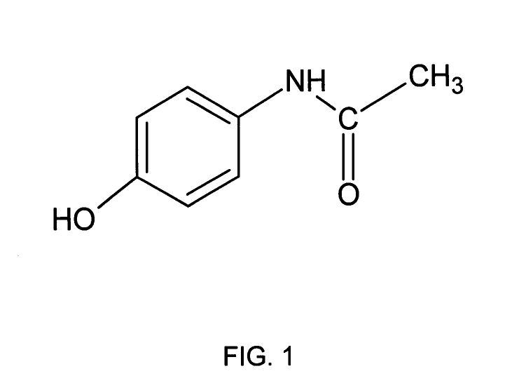

FIG. 2 shows the approximate relative distribution of the products of acetaminophen metabolism under normal conditions in an adult in its known metabolic pathways;

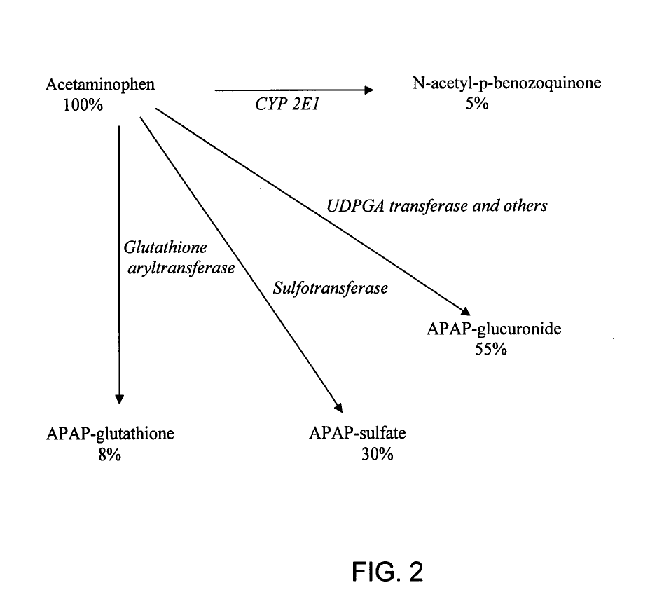

FIG. 3 shows the chemical structure of acetaminophen and its Phase II metabolite after glucuronidation via the UDP glucuronosyl transferase enzyme;

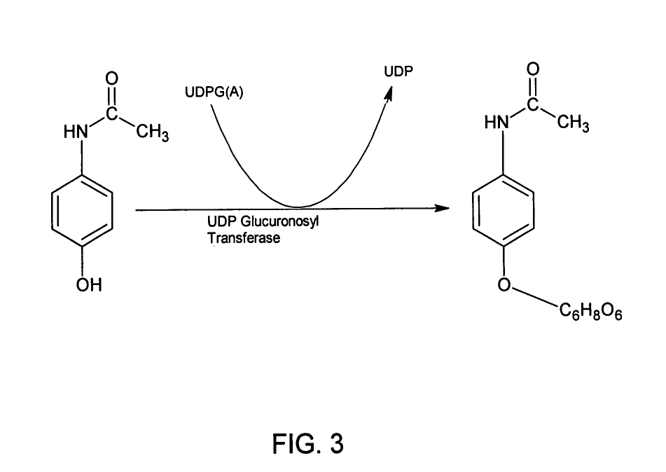

FIG. 4 shows the chemical structure of acetaminophen and its Phase II metabolite after sulfonation via the sulfotransferase enzyme;

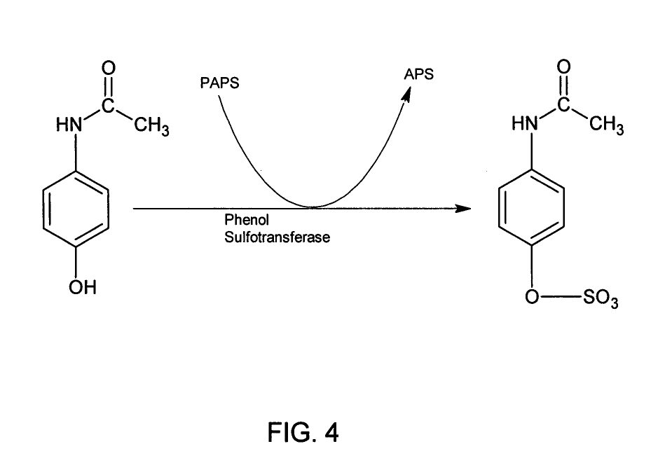

FIG. 5 shows the chemical structure of acetaminophen and its Phase I metabolites, involving the cytochrome P450 system;

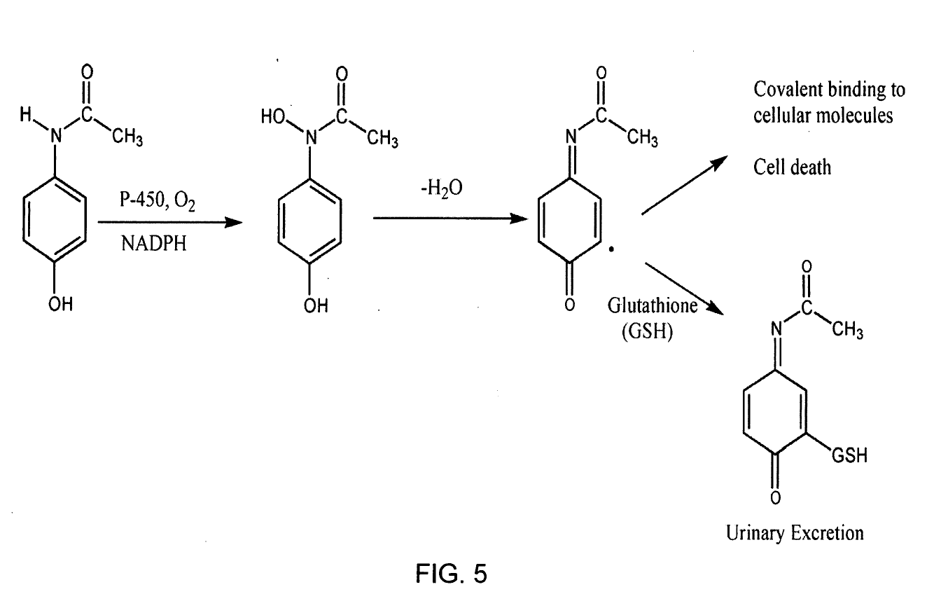

FIG. 6 shows the enzymatic steps involved in the in vivo synthesis of uridine diphosphoglucose, and its interaction with acetaminophen to form a glucuronide conjugate;

FIG. 7 shows the enzymatic steps involved in the in vivo synthesis of phosphoadenosine phosphosulfate, and its interaction with acetaminophen to form a sulfonate conjugate; and

FIG. 8 presents mortality curves of two groups of animals comparing survival over time after the administration of either APAP/UDPG or APAP/saline.

### DETAILED DESCRIPTION OF THE INVENTION

Acetaminophen can cause liver toxicity when its normal Phase II metabolic pathways are overwhelmed. If this happens, excessive amounts of the drug are metabolized through the Phase I pathway, the cytochrome P-450 enzyme system. This may result in liver damage and other deleterious effects, because the product of this Phase I pathway (NAPQI) is toxic. The standard approach to treatment and even prevention of these problems involves supplying adequate amounts of a glutathione substitute to permit detoxification of NAPQI, the toxic product resulting from Phase I metabolism. The present invention is based, in part, on the discovery that enhancing the glucuronidation or sulfonation metabolic pathways for acetaminophen (and any other similarly metabolized chemical compound) will reduce the formation of toxic intermediate compounds via the Phase I cytochrome P-450 system. Glucuronidation or sulfonation of the acetaminophen molecule removes it as a substrate for oxidation via the cytochrome enzyme system. However, common belief by those skilled in the art is that UDPGA is not a limiting factor in the metabolism of drugs. Experts in the field have stated that “[d]ue to the reasonably high concentrations [of UDPGA] in the liver (˜250 μM), cofactor depletion is probably not an issue”. (Bjornsson, T. D., et al., D.M.D. 2003; 31:815-32). This conventional view notwithstanding, it has now been discovered that providing exogenous uridine diphosphate-based donors, and/or phosphoadenosine-phosphosulfate-based donors, can in fact help to prevent the toxicity associated with the ingestion of excessive amounts of acetaminophen.

In Europe, UDPG has been suggested as a treatment for several conditions, including hepatic disease (Tarantino, G., et al., Riv. Eur. Sci. Med. Farmacol. 1990; 12:109-17), porphyria (Okolicsanyi, L., et al., Clin. Ter. 1980; 94:687-94), amanita phalloides mushroom poisoning (Delfino, U., et al., Curr. Prob. Clin. Biochem. 1977; 7:167-84), and hyperbilirubinemia in newborn infants (Salazar De Sousa, J., et al., Minerva Pediatr. 1975; 27:1162-5). UDPG has been available parenterally (Toxepasi-4000-Roche), and a mixture of UDPG, glutathione and vitamin B-12 (Toxepasi Complex) has been available orally for treatment of newborn hyperbilirubinemia, chronic hepatitis and liver cirrhosis. It has also been examined as a rescue agent in cancer therapy after the administration of 5-Fluorouracil (Codacci-Pisanelli et al., Oncology, 2002; 62:363-70). It should be emphasized that UDPG, and not UDPG-A, was utilized in Europe.

Neither the Europeans nor anyone else has suggested using UDPG-A or its precursors for the treatment of acetaminophen poisoning.

Nor has there been any suggestion to use UDPG-A or its precursors as a preventative drug for any indication.

There has also been no suggestion to use UDPGA or its precursors as either a preventative or therapeutic agent.

Similarly, there has been no suggestion to use the sulfonation donors, including PAPS and APS for prevention or therapy of liver toxicity.

Glucuronosyl transferases (UDPGA-T) are a group of hepatic enzymes that transfer glucuronic acid from uridine diphospho-glucuronic acid (also known as diphospho-glucuronide) (UDPGA) to an acceptor. This is the principal Phase II enzyme system in the liver. UDPGA-T is found in many plant and animal species. It has five known polymorphisms that have considerable selectivity. UDPGA is the natural cofactor/substrate for glucuronosyl transferase. The common opinion is that UDPGA is not a limiting factor in the metabolism of drugs. An article representing the views of the Pharmaceutical Research and Manufacturers of America (PhRMA) states in reference to UDPG and UDPGA systems: “[D]ue to the reasonably high concentrations (of UDPGA) in the liver (˜250 μM), cofactor depletion is probably not an issue”. (Bjornsson, Callaghan et al. 2003).

Regardless of this “conventional wisdom”, it has now been discovered that providing glucuronidation cofactors, and/or sulfonation cofactors, can in fact help to prevent the toxicity associated with the ingestion of excessive amounts of acetaminophen. This discovery is supported by the fact that there is some evidence that UDPGA is depleted in hepatic toxic states caused by excessive APAP (Hjelle, 1986). The occurrence of UDPGA deficiencies may also be inferred by a critical analysis of the metabolite concentrations measured in human studies (Davis, M., Simmons, J., Harrison, N. G., Williams, R. Quart. J. Med, 1976, 45:181-191), (Slattery, J. T., Wilson, J. M., Kalhom, T. T., Nelson, S. Clin. Pharm. & Ther. 1987; 41:413-8)) and (Forest, A. H., Clements, J. A. Preesott, L. F. Clin. Pharmacokinetics, 1982, 7:93-107).

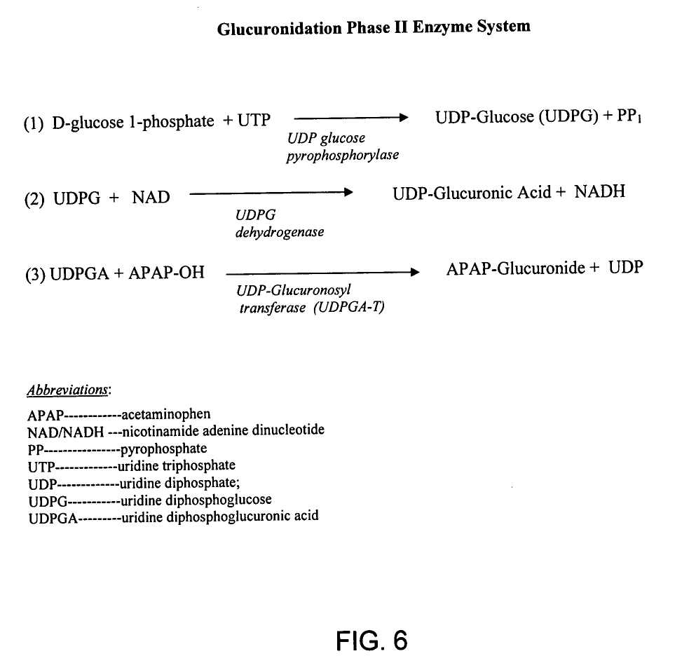

Glucuronide synthesis and conjugation is shown in FIG. 6. Glucose-1 phosphate combines with uridine triphosphate (UTP) in the presence of UDP glucose pyrophosphorylase to yield uridine diphosphoglucose (UDPG). UDPG dehydrogenase catalyzes the formation of UDP-glucuronic acid (UDPGA) in the presence of nicotinamide adenine dinucleotide (NAD⁺). UDPGA is then available to combine with acetaminophen (APAP-OH) to form its conjugate, APAP-glucuronide. The reduced form of NAD⁺ (NADH) is a “potent competitive inhibitor (K of 27 μM) of its oxidized form, NAD⁺ (Km of 424 μM) in the UDP-glucose dehydrogenase reaction” (Hjelle, J. J., J. Pharm. & Exp. Ther. 1986, 237:750-56). In a normal oxygenated state, NAD⁺ is the predominant nucleotide.

There is some suggestion that UDPGA is depleted after the administration of acetaminophen in a time-dependent and dosage dependent fashion (Hjelle 1986). That the glucuronidation pathway can be overloaded is supported by the fact that peak excretion of acetaminophen-glucuronide occurs before levels of acetaminophen start to decline, and larger doses of acetaminophen generate progressively lesser amounts of acetaminophen-glucuronide (Slattery, J. T., Levy, G., Clin. Pharm. & Ther. 1979; 25:184-195). Thus it appears that the glucuronidation pathway may be depleted of adequate substrate or cofactors as the liver is exposed to increasing amounts of acetaminophen. However, heretofore, no one has suggested that UDPG-A, or its salts or derivatives, be given to combat acetaminophen toxicity.

It has been shown that acetaminophen detoxification competes with gluconeogenesis for the available UDPG, providing further evidence that saturation of the UDPG/glucuronidation pathway may occur. UDPG is used by the enzymes glycogenin and glycogen synthase to manufacture glycogen. This pathway is competitive with the utilization of UDPG to form UDPGA for glucuronidation to detoxify chemical compounds. Thus, conditions or substances that drive UDPG towards glycogen synthesis deplete the availability of UDPGA for glucuronidation of toxic substances. Fasting is one example in which gluconeogenesis is increased at the expense of UDPGA formation (Price, V. F., Jollow, D. J., Biochem. Pharmacol. 1988, 15:1067-75). Under experimental conditions, acetaminophen is known to deplete the concentrations of UDPGA in rats, “the extent of depletion and the time required for recovery to pre-drug levels were dependent on the dose of acetaminophen administered. The amount of UDPGA required for the glucuronidation of a therapeutic dose was nearly equal to the total content of UDPGA in the liver; after a toxic dose, the UDPGA demand was over 100-fold greater than the normal basal level” (Price, V. F., Jollow, D. J., Xenobiotica 1984, 14:553-9). These two facts (i.e., competition between the gluconeogenesis pathway and the glucuronidation pathway and the utilization of acetaminophen to measure glucuronidation) have been combined to develop an in vivo assay for gluconeogenesis by determining the amount of acetaminophen glucuronidation that occurs under experimental conditions. (Schwenk W. F., Kahl, J. C., Am. J. Physiol. 1996; 271:E529-34; Rother, K. I., Schwenk, W. F., Am. J. Physiol. 1995; 269:E766-73). In the setting of galactosamine-depleted UDPG in rats, glucuronidation of acetaminophen is decreased. (Gregus, Z., et al., Drug Metab. Dispos. 1988; 16:527-33).

It has now been discovered that replenishment of UDPG or UDPGA can restore the level of acetaminophen glucuronidation. Therefore, one aspect of this invention is to provide uridine diphosphoglucuronic acid (UDPGA), or its acids, salts or precursors to counteract the depletion of UDPGA in the liver, thereby accelerating the rate of acetaminophen-glucuronide formation, and lessening the amount of acetaminophen available for oxidation via Phase I metabolism to NAPQI. The same treatment can be administered to an individual who has been exposed to any chemical compound capable of metabolism through glucuronidation.

The use of UDPGA may be preferable over its precursor, UDPG, in conditions that favor a reduced chemical state in the liver. The transformation of UDPG to UDPGA requires both UDPG availability and also oxidized NAD⁺. As noted, UDPG is converted by UDPG dehydrogenase to UDP-glucuronic acid (UDPGA). UDPGA is the substrate for transfer of glucuronic acid to acetaminophen, as shown in FIG. 6. An energy depleted state (e.g., lack of ATP with many reducing electrons available), such as that which occurs after the administration of toxic (and perhaps non-toxic) levels of acetaminophen, would drive NAD to NADH. NADH inhibits acetaminophen glucuronidation (Hjelle, J. J., J. Pharm. & Exp. Ther. 1986; 237:750-56; Dills, R. L., et al., Drug Metab. Dispos. 1987; 15:281-8). Thus, in the setting of toxic levels of a chemical compound such as acetaminophen, it may be advantageous to provide exogenous UDPGA or its salts, because the conjugation and detoxification will be less NAD⁺-dependent.

It should be appreciated that other compounds are potentially amenable to detoxification using this approach. Such compounds can include aspirin, caffeine and steroid hormones, (e.g., testosterone, progesterone and estradiol, as well as their synthetic analogues, including Tamoxifen and other anti-estrogen compounds). Contraceptive agents may also be amenable to this approach for detoxification treatment. Generally, UDPGA or its salts can be administered after exposure to any pharmaceutical compound or chemical agent that is susceptible to conjugation by glucuronidation.

If the liver is not in a chemically-reduced state, then UDPG, UDPGA, UDP, UTP, and salts, acids, and mixtures thereof, can all be used either to prevent or treat potential liver toxicity from chemical compounds such as acetaminophen. Preventive therapy can include the co-administration of any of the aforementioned compounds with acetaminophen in therapeutic doses. The added preventative agents of the present invention can offset the cumulative toxicity of repeated or prolonged exposure to acetaminophen, even when individual doses are considered to be non-toxic.

In cases of oral administration, UTP and UDP may be preferred over the other forms because of their greater resistance to premature degradation in the acid environment of the stomach.

Furthermore, any of the aforementioned compositions of the present invention can also be co-administered with aspirin, caffeine, steroid hormones, and any drug that can be metabolized via the glucuronidation pathway. Included among the steroid hormones are substances such as testosterone, progesterone and estradiol, as well as their synthetic analogues, including Tamoxifen and other anti-estrogen compounds. Contraceptive agents can also be included as compounds co-administered with the compositions of the present invention. In general, any of the compositions of the present invention can be co-administered with any pharmaceutical compound or chemical agent that is susceptible to metabolism via glucuronidation. The toxic effects of many non-pharmaceutical compounds can potentially be reduced by the use of the compositions of the present invention, including, for example, aniline and benzene.

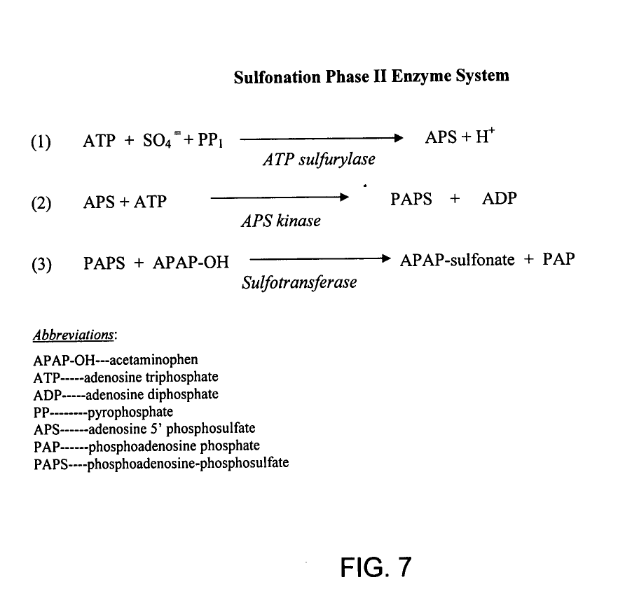

The second important pathway of Phase II metabolism is the sulfonation pathway. As shown in FIG. 7, the sulfonation pathway requires more high energy phosphate (ATP), and the sulfo-transferase enzymes may be present in lower concentration than those of the glucuronidation pathway. However, the sulfonation pathway is just as prone to competition with other systems as the glucuronidation pathway is subject to competition with gluconeogenesis. Thus, augmenting the sulfonation pathway by supplying one or more of its precursors such as adenosyl-5′-phosphosulfate (APS) and especially 3-phospho-adenosyl-5′-phosphosulfate (PAPS), in a manner analogous to that described above for UDPG, will result in increased metabolism through the sulfonation pathway. These cofactors can be used as an alternative to the UDPG group of substances, or in conjunction with the UDPG group of substances, as a preventative or therapeutic agent.

By way of example but not limitation, an alternative approach for treating potentially toxic exposure to a chemical compound such as acetaminophen is to co-administer compositions including UDPG, UDPGA, UTP, UDP, PAPS, APS and their salts or mixtures thereof, with sulfhydryl substrates, including NAC, methionine, L-cysteine, diallyl sulfide, and diallyl sulfone. This two-fold approach prevents the oxidative damage associated with metabolism via the cytochrome P-450 pathway by reducing its substrates on the one hand, and by detoxifying its products on the other.

The dosing range of the compositions of the present invention can be from about 0.1 to 10 moles, about 0.5 to 2 moles, or even about 1 mole, per mole of acetaminophen ingested. Given the approximate molecular weights of the compounds (UDPG MW=566, acetaminophen MW=151), a molar ratio of 1:1 yields approximately 566 mgs of UDPGA to 151 mg of acetaminophen. Smaller doses can be administered. For PAPS, whose molecular weight is approximately 504, slightly smaller gravimetric amounts can be used.

For treatment of a hepatotoxic exposure to a pharmaceutical or chemical compound (such as acetaminophen), relatively higher doses of UDPG, UDPGA, UTP, UDP, and their salts, acids or mixtures thereof, can be used. The toxic doses of UDPGA and PAPS in humans are not known.

The compositions of the invention can be administered by oral, rectal, vaginal, topical, mucosal, or parenteral means. Oral forms can include a tablet, capsule, gel wafer, chewable tablet, caplet, melt-away, buffered effervescent granules, solution, lyophilized solution, suspension, lyophilized suspension, syrup or elixir.

### EXAMPLE

### Efficacy of Uridine Diphsopho Glucose (UDPG) to Prevent Acetaminophen (APAP)-Induced Toxicity in Mice

To validate the concept that UDPG can mitigate the toxicity of APAP, a dose-response of APAP toxicity was established for C57B16/J male mice weighing approximately 20 grams each between the ages of 6 to 10 weeks. In prior experiments in the laboratory, the LD₆₀, of APAP was determined to be about 600 mgs/kg. The molecular weight of APAP is 151; therefore, the LD₆₀, was about 4 mMol/Kg.

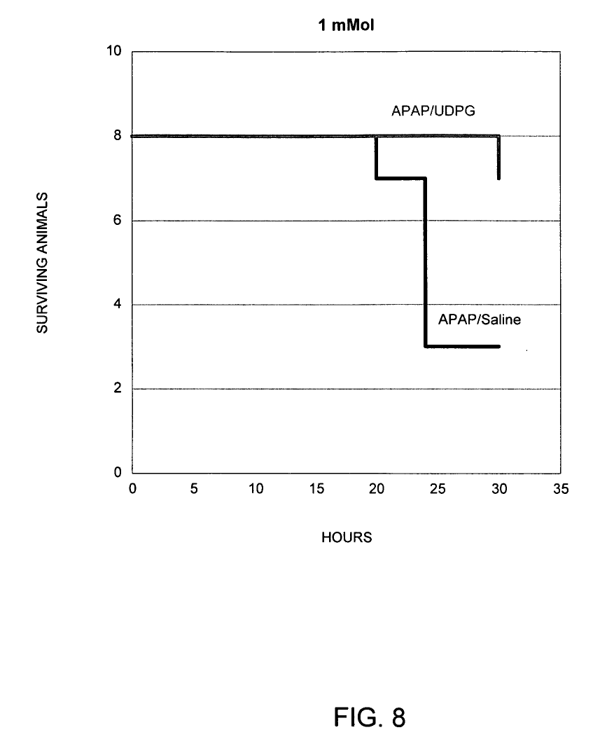

In this experiment, the animals were transferred to newly cleaned cages with mesh floors and fasted for 24 hours. They were divided into two groups of eight mice per group. The mice were allowed free access to water and allowed food 8 hours after the administration of the drugs. Sixteen hours after the fast began, four mMol/kg (600 mg/kg) of APAP, previously dissolved in warm saline to a concentration of 15 mg/ml, was administered intra-peritoneally (IP). At the same time, each animal received approximately one mMol (600 mg) of UDPG by subcutaneous injection. A control group received an identical amount of APAP and an equivalent volume (0.2 ml) of physiological saline. Animals were inspected every two hours for 30 hours to ascertain survival. The data were plotted for Kaplan-Myer analysis. Mortality curves are presented in FIG. 8.

Three of the eight APAP/saline-treated animals (control group) survived (37.5%) and 100% of the APAP/UDPG treated animals survived at 24 hours. A single animal died at 30 hours. It can be concluded that UDPG was completely effective in preventing APAP-induced mortality at these doses within the designated 24 hour test period.

### MODIFICATIONS

While the invention has been particularly shown and described with reference to preferred embodiments thereof, it will be understood by those skilled in the art that other changes in form and details may be made therein without departing from the spirit and scope of the invention. In particular, it will be clear to those of skill in the art that the foregoing compounds will function individually or in any combination in a similar manner for the prevention or treatment of any chemical compound that is susceptible to glucuronide conjugation and/or sulfonate conjugation in the body.

What is claimed is:

1. A pharmaceutical composition comprising (i) a xenobiotic substance that is hydroxylated or can be hydroxylated and (ii) a glucuronidation donor for yielding a xenobiotic glucuronide, wherein the glucuronidation donor comprises at least one from the group consisting of: uridine diphosphoglucuronic acid (UDPGA); precursors thereof; salts thereof; and mixtures thereof.

2. A pharmaceutical composition according to claim 1 wherein the xenobiotic substance comprises acetaminophen and further wherein the xenobiotic glucuronide comprises acetaminophen glucuronide (APAP-G).

3. A pharmaceutical composition according to claim 2 wherein the composition further comprises a compound selected from the group consisting of: N-acetylcysteine (NAC); methionine; or inhibitors of cytochrome P-450 metabolism.

4. A pharmaceutical composition according to claim 2 wherein the composition further comprises a sulfonation donor, wherein the sulfonation donor comprises one from the group consisting of: phosphoadenosine-phosphosulfate (PAPS); precursors thereof; salts thereof; and mixtures thereof.

5. A pharmaceutical composition according to claim 4 wherein the composition further comprises a compound selected from the group consisting of: N-acetylcysteine (NAC); methionine; glutathione donors; sulfation cofactors; and inhibitors of cytochrome P-450 metabolism.

6. A pharmaceutical composition according to of claim 2 wherein the composition is adapted for administration to a patient in a mode selected from the group consisting of: oral; rectal; vaginal; transdermal; and parenteral.

7. A pharmaceutical composition comprising (i) a xenobiotic substance that is hydroxylated or can be hydroxylated and (ii) a sulfonation donor for yielding a xenobiotic sulfonate, wherein the sulfonation donor comprises at least one from the group consisting of: phosphoadenosine-phosphosulfate (PAPS); precursors thereof; salts thereof; and mixtures thereof.

8. A pharmaceutical composition according to claim 7, wherein the xenobiotic substance comprises acetaminophen and further wherein the xenobiotic sulfonate comprises acetaminophen sulfonate (APAP-S).

9. A pharmaceutical composition according to claim 8, wherein the composition further comprises a compound selected from the group consisting of: N-acetylcysteine (NAC); methionine; glutathione donors; and inhibitors of cytochrome P-450 metabolism.

10. A pharmaceutical composition according to of claim 8 wherein the composition is adapted for administration to a patient in a mode selected from the group consisting of: oral; rectal; vaginal; transdermal; and parenteral.

11. A method for preventing liver damage in a patient caused by the ingestion of an xenobiotic substance, that is hydroxylated or can be hydroxylated, comprising administering a therapeutically effective dose of a glycuronidation donor to the patient, wherein the glucuronidaton donor comprises one from the group consisting of: uridine diphosphoglucuonic acid (UDPGA); precursors thereof; salts thereof; and mixtures thereof.

12. A method according to claim 11 wherein the xenobiotic substance comprises acetaminophen.

13. A method according to claim 12 wherein the glycuronidation donor is administered to the patient after the acetaminophen has been administered.

14. A method according to claim 12 wherein the glycuronidation donor is administered to the patient at the same time that the acetaminophen is administered.

15. A method according to claim 14 wherein the glycuronidation donor and the acetaminophen are administered as a single dosage.

16. A method according to claim 12 wherein the method further comprises administering a compound selected from the group consisting of: N-acetylcysteine (NAC); methionine; glutathione donors; sulfation cofactors; and inhibitors of cytochrome P-450 metabolism.

17. A method according to claim 12 wherein the method further comprises administering a therapeutically effective dose of a sulfonation donor to the patient, wherein the sulfonation donor comprises one from the group consisting of: phosphoadenosine-phosphosulfate (PAPS); precursors thereof; salts thereof; and mixtures thereof.

18. A method according to claim 17 wherein the method further comprises administering a compound selected from the group consisting of: N-acetylcysteine (NAC); methionine; glutathione donors; sulfation cofactors; and inhibitors of cytochrome P-450 metabolism.

19. A method according to claim 12 wherein the route of administration of the glucuronidation donor is selected from the group consisting of: oral; rectal; vaginal; transdermal; and parenteral.

20. A method for preventing liver damage in a patient caused by the ingestion of an xenobiotic substance that is hydroxylated or can be hydroxylated, comprising administering a therapeutically effective dose of a sulfonation donor to the patient, wherein the sulfonation donor comprises one from the group consisting of: phosphoadenosine-phosphosulfate (PAPS); precursors thereof; salts thereof; and mixtures thereof.

21. A method according to claim 20 wherein the xenobiotic substance comprises acetaminophen.

22. A method according to claim 21 wherein the sulfonation donor is administered to the patient after the acetaminophen has been administered.

23. A method according to claim 21 wherein the sulfonation donor is administered to the patient at the same time that the acetaminophen is administered.

24. A method according to claim 23 wherein the sulfonation donor and the acetaminophen are administered as a single dosage.

25. A method according to claim 21 wherein the method further comprises administering a compound selected from the group consisting of: N-acetylcysteine (NAC); methionine; glutathione donors; glucuronidation cofactors; and inhibitors of cytochrome P-450 metabolism.
